(19) United States
(12) Patent Application Publication
(10) Pub. No.: US 2017/0224840 A1
(43) Pub. Date: Aug. 10, 2017

(54) Drug Delivery System Comprising A Cancer Stem Cell-Targeted Carbon Nanotube, Preparation and Use Thereof

(71) Applicant: Institute of Pharmacology and Toxicology Academy of Military Medical Sciences P.L.A. China, Beijing (CN)

(72) Inventors: Hongjuan YAO, Beijing (CN); Yingge ZHANG, Beijing (CN); Lan SUN, Beijing (CN); Yan LIU, Beijing (CN)

(73) Assignee: Institute of Pharmacology and Toxicology Academy of Military Medical Sciences P.L.A. China, Beijing (CN)

(21) Appl. No.: 15/502,954

(22) PCT Filed: Aug. 11, 2015
(86) PCT No.: PCT/CN2015/086587

§ 371 Date: Feb. 9, 2017

(30) Foreign Application Priority Data
Aug. 11, 2014 (CN) 201410391787.9

(51) Int. Cl. A61K 9/50; A61K 9/51; A61K 31/351

(52) U.S. Cl. CPC A61K 47/48861; A61K 47/48015; A61K 9/5161; A61K 31/351; A61K 9/5192; A61K 47/48869; A61K 47/4823

(57) ABSTRACT

The present invention relates to a drug delivery system, comprising: a drug-loaded carbon nanotube formed by a carbon nanotube and a drug molecule adsorbed on the surface of the carbon nanotube, a modifying material capable of enhancing water solubility and biocompatibility of the drug delivery system, and a targeting molecule. The present invention further relates to preparation and use of the drug delivery system. The present invention provides a new strategy for selectively targeting and effectively eliminating cancer stem cells, which is conducive to fundamentally preventing recurrence and metastasis of a cancer induced by cancer stem cells.

### TECHNICAL FIELD

The present invention relates to the field of cancer stem cell-targeted therapy, especially to a drug delivery system comprising a cancer stein cell-targeted carbon nanotube, preparation and use thereof.

### BACKGROUND ART

Tumors are abnormal pathologic changes formed by clonal abnormal proliferation of a certain cell in local tissue, which is caused by loss of normal control of growth of the cell in genetic level under the action of a variety of carcinogenic factors. The greatest difficulties in cancer treatment are resistance, recurrence and metastasis. According to the theory of tumor stem cells, drug-resistance, recurrence and metastasis are due to the presence of tumor stem cells.

Tumor stem cells are special cancer cells with self-renewal and multi-directional differentiation potential in tumor tissues, and are directly related to tumor occurrence, recurrence and metastasis. Tumor stem cells show strong resistance to conventional chemotherapeutic drugs, show tolerance to radiation therapy, and tumor stem cells are of high tumorigenicity and high invasion and metastasis. After surgery, drug therapy, radiotherapy and so on, most of differentiated tumor cells in cancer patients may be killed or inhibited, but a small amount of residual tumor stem cells in body may act as seeds and sources and play a decisive role in proliferation, growth, invasion, metastasis and recurrence of tumors. Moreover, clinical studies have shown that tumor stem cells are closely related to tumor metastasis, recurrence and prognosis.

Drug resistance is one of the characteristics of tumor stem cells, and the drug resistance mechanisms are manifested in many aspects: (1) tumor stein cells exist in the center of tumor tissue, and general anti-tumor drugs are difficult to enter the tumor tissue, thus even a drug with anti-stem cell effect is difficult to kill them; (2) tumor stem cells are usually in quiescent period, rarely in differential and proliferation period, so that they are not sensitive to many anti-tumor drugs, and can hardly be killed by cycle-specific conventional anti-tumor drugs; (3) ABC transporters (ATP-binding cassette transporters) family proteins on the tumor stem cell membrane are over-expressed, so that tumor stem cells have natural multidrug resistance; (4) tumor stem cells are able to generate drug resistance and resistance to chemotherapy through high expression of apoptosis-inhibiting genes and low expression of apoptosis-prompting genes; that is, endogenous drug-resistance is a congenital self-protection mechanism of tumor stem cells; (5) the high efficiency of DNA repair in tumor stem cells is an important mechanism for resistance to chemotherapy and radiotherapy, and is also an important reason that tumor stem cells develop the resistance to chemotherapeutic drugs and radiotherapy rays; in addition, tumor stem cells are often localized in a hypoxic niche environment which may act as a barrier to protect tumor stem cells from exposure to chemotherapeutic agents and radiation, thereby improving their ability to escape. The above-mentioned mechanisms of drug resistance make tumor stem cells survive after conventional tumor therapy, and the tumor stein cells have function of self-renewal and multi-directional differentiation, thus under appropriate conditions, tumor stem cells can re-proliferate and lead to tumor recurrence and metastasis. Tumor resistance, metastasis and recurrence may possibly be avoided by killing the tumor stem cells, to achieve the cure of tumor. Therefore, the tumor stem cells have become a new target for tumor therapy, and the development of cancer stem cell targeted therapy strategy has important clinical value.

Modified carbon nanotubes have excellent properties of transmembrane, high drug-loading capacity, controlled and sustained drug release, easy functional modification, good biocompatibility and long-time of in vivo circulation, and they can be excreted from body through renal metabolism and urine, therefore they have unparalleled advantages in drug delivery system. In recent years, some research reports show that drug delivery systems with carbon nanotube are started to be used in animal levels, and encouraging results are achieved. However, there is no report about use of carbon nanotubes as a tumor stem cell targeted carrier, and it is unknown whether carbon nanotubes can deliver drug molecules to tumor stem cells.

### Contents of the Invention

After long period of experimentation, the inventors of the present invention have surprisingly found that carbon nanotubes can be used as a carrier material to load drug molecules, and after being underwent a series of modifications, they can be used for selectively targeting and effectively eliminating cancer stem cells. The present invention is completed based on these findings.

The first aspect of the present invention relates to a drug delivery system, comprising: a drug-loaded carbon nanotube formed by a carbon nanotube and a drug molecule adsorbed on the surface of the carbon nanotube, a modifying material capable of enhancing water solubility and biocompatibility of the drug delivery system, and a targeting molecule.

In one embodiment of the invention, the drug molecule is loaded onto the surface of the carbon nanotube by hydrophobic interaction.

In one embodiment of the invention, the modifying material is coated on the surface of the drug-loaded carbon nanotube by electrostatic self-assembly, and so as to obtain a modified drug-loaded carbon nanotube.

In one embodiment of the invention, the targeting molecule is coated on the surface of the modified drug-loaded carbon nanotube by electrostatic self-assembly.

In one embodiment of the present invention, the carbon nanotube carries a negative charge.

In one embodiment of the invention, the drug molecule carries a negative charge.

In one embodiment of the invention, the modifying material carries a positive charge.

In one embodiment of the invention, the targeting molecule carries a negative charge.

In one embodiment of the present invention, the drug-loaded carbon nanotube has a particle size of 130-200 nm, preferably 130-180 nm, and particularly preferably 130-160 nm.

In one embodiment of the present invention, the drug-loaded carbon nanotube has a drug-loading capacity of 10 to 40% by weight, for example 15 to 30% by weight.

In one embodiment of the invention, the drug delivery system has a particle size of 150-400 nm, for example 200-350 nm, for example 220-300 nm.

In an embodiment of the present invention, the carbon nanotube is single-walled carbon nanotube or multi-walled carbon nanotube.

In one embodiment of the present invention, the carbon nanotube has a length of 100 to 1000 nm, preferably 150 to 400 nm.

In one embodiment of the present invention, the carbon nanotube has an inner diameter of 1 to 3 nm, preferably 1 to 2 nm.

In one embodiment of the present invention, the carbon nanotube is an oxidized carbon nanotube.

In one embodiment of the present invention, the oxidized carbon nanotube has a particle size of 100 to 200 nm, preferably 100 to 150 nm, and particularly preferably 130 to 150 nm.

In one embodiment of the invention, the drug molecule is a drug capable of specifically killing a tumor stem cell, such as salinomycin or a pharmaceutically acceptable salt or derivative thereof, parthenolide, sulforaphene, curcumin, resveratrol, metformin and so on.

In one embodiment of the invention, the modifying material is selected from the group consisting of a polymer macromolecule, a natural polysaccharide, a surfactant, an aromatic ring compound and a biological macromolecule and so on. The modifying material is coated on the surface of drug-loaded carbon nanotube by non-covalent modification method. Non-covalent modification method is simple and easy to operate, would not destroy the complete structure of carbon nanotube, and would not affect the mechanical and electrical properties of carbon nanotube. The non-covalent modification mainly utilizes electrostatic attraction force, π-π stacking force, van der Waals force and hydrophobic force to coat the modifying material onto the wall of carbon nanotube. The hydrophilic moieties of the modifying material act with water or polar solvent to prevent agglomeration of CNTs and make them well dispersed in the solvent. The modifying material of the invention is selected from the group consisting of chitosan, polyethylene glycol, a pluronic block polymer, cellulose, and preferably, the modifying material is chitosan. Chitosan is a natural macromolecular cationic polymer obtained by deacetylation of chitin, and is the only basic polysaccharide among polysaccharides. In one embodiment of the invention, chitosan is used for non-covalent modification of the carbon nanotube, which can effectively improve the water dispersibility and biocompatibility of the carbon nanotube.

In one embodiment of the invention, the targeting molecule is a molecule capable of specifically targeting a cancer stem cell, for example selected from the group consisting of molecules capable of specifically targeting gastric cancer stem cells, breast cancer stem cells, endometrial cancer stem cells, lung cancer stem cells or colorectal cancer stem cells.

In one embodiment of the invention, the targeting molecule is selected from molecules that are capable of specifically binding to a cellular marker on the surface of a cancer stem cell, such as a molecule capable of binding specifically to CD44, CD24, CD133, CD34, CD166 or EpCAM, for example is hyaluronic acid, P-selectin, or an antibody, for example a monoclonal antibody, capable of specifically binding to the cellular marker.

A second aspect of the present invention relates to a method for preparing the drug delivery system according to any one of items of the first aspect of the invention, comprising the following steps:

(1) loading the drug molecule onto the surface of the carbon nanotube by a non-covalent interaction (e.g., π-π stacking interaction or hydrophobic interaction) to obtain the drug-loaded carbon nanotube;

(2) coating the modifying material onto the surface of the drug-loaded carbon nanotube to obtain the modified drug-loaded carbon nanotube;

(3) adsorbing the targeting molecule to the surface of the modifying material to obtain the drug delivery system;

preferably, the method further comprising: prior to step (1), a step of subjecting the carbon nanotube to oxidation treatment with a concentrated acid (e.g., concentrated nitric acid, concentrated sulfuric acid, or a mixture thereof).

In one embodiment of the invention, the modifying material is coated on the surface of the drug-loaded carbon nanotube by electrostatic self-assembly.

In one embodiment of the invention, the targeting molecule is coated on the surface of the modified drug-loaded carbon nanotube by electrostatic self-assembly.

In one embodiment of the present invention, the preparation method comprises the steps of:

1) a drug is dissolved in methanol to prepare a drug solution, the resultant drug solution is mixed with carbon nanotubes, subjected to ultrasonic treatment, dried, followed by addition of a buffer solution (e.g., a phosphate buffer solution, Tris-HCl buffer solution), subjected to a further ultrasonic treatment, collected with microfiltration membrane, washed and dried to obtain the drug-loaded carbon nanotube;

2) the drug-loaded carbon nanotube obtained in the step 1) is added to an aqueous solution of the modifying material, subjected to ultrasonic treatment, washed by a centrifugation-ultrasonic treatment-centrifugation method to obtain the modified drug-loaded carbon nanotube;

3) the modified drug-loaded carbon nanotube obtained in step 2) is added to an aqueous solution of the targeting molecule, subjected to ultrasonic treatment, washed by a centrifugation-ultrasonic treatment-centrifugation method to obtain the drug delivery system.

Preferably, the method further comprises a step of subjecting the carbon nanotubes to an oxidation treatment prior to step 1).

Preferably, the oxidation treatment is carried out by dispersing the carbon nanotubes in a concentrated sulfuric acid/concentrated nitric acid mixed acid, subjecting to ultrasonic treatment, filtering, washing with water, removing oxidization debris with NaOH solution, washing with water, and freeze-drying.

In a specific embodiment of the present invention, the drug and the carbon nanotubes of step 1) are in a weight ratio of 1-10:1, preferably 2-5:1, particularly preferably 3:1.

In a specific embodiment of the present invention, in step 1), the ultrasonic treatment is performed each time for 1 to 8 hours, preferably 6 hours.

In a specific embodiment of the present invention, in step 1), the microfiltration membrane collects oxidized carbon nanotubes having a particle size of less than 0.1 μm.

In a specific embodiment of the invention, the modifying material and the oxide carbon nanotubes in step 2) are in a weight ratio of 1-10:1, preferably 5.1.

In a specific embodiment of the present invention, in step 2), the ultrasonic treatment is performed for 0.5 to 2 hours, preferably for 30 minutes.

In a specific embodiment of the present invention, the targeting molecule used in step 3) and the product obtained in step 2) are in a weight ratio of 1-10:1, preferably 2:1.

In a specific embodiment of the present invention, in step 3), the ultrasonic treatment is performed for 0.5 to 2 h, preferably 30 min.

In a specific embodiment of the present invention, in the oxidization step, the concentrated sulfuric acid and the concentrated nitric acid are in a volume ratio of 1-5:1, preferably 3:1.

In a specific embodiment of the present invention, in the oxidization treatment step, the carbon nanotubes and the mixed acid are in a ratio of 1-3:1 (m/v), preferably 1:1 (m/v).

In a specific embodiment of the present invention, in the oxidization treatment step, the ultrasonic treatment is performed for 8-24 h, preferably 12 h.

In a specific embodiment of the present invention, in the oxidization treatment step, the oxidized carbon nanotubes collected by filtration have a particle diameter of more than 0.1 μm, preferably 0.10 to 0.45 μm.

In the carbon nanotube drug delivery system of the invention, the drug molecule, the modifying material and the targeting molecule are all supported on the carbon nanotube by non-covalent binding method; in comparison with covalent binding assembly methods, the preparation of the present invention is obviously simplified and has promising application prospects.

The third aspect of the invention relates to a pharmaceutical composition comprising the drug delivery system according to any one of items of the first aspect of the present invention, and a pharmaceutically acceptable carrier or excipient.

A fourth aspect of the invention relates to a use of the drug delivery system according to any one of items of the first aspect of the present invention in manufacture of a medicament for prophylaxis or treatment of a malignant tumor or inhibition of growth, proliferation, migration or invasion of a tumor.

In one embodiment of the invention, the malignant tumor is a malignant tumor derived from epiblast, for example, a tumor selected from the group consisting of brain tumor, stomach cancer, lung cancer, pancreatic cancer, colorectal cancer, breast cancer, prostate cancer, endometrial cancer, ovarian cancer and leukemia.

The present invention also relates to a method of preventing or treating a malignant tumor or inhibiting growth, proliferation, migration or invasion of a tumor, comprising: administering to a subject in need thereof the drug delivery system according to any one of items of the first aspect of the present invention, or the pharmaceutical composition according to any one of items of the third aspect of the present invention.

In one embodiment of the invention, the subject is a mammal, such as a bovine, an equine, a goat, a porcine, a canine, a feline, a rodent, a primate animal; preferably, the subject is a human.

In one embodiment of the invention, the malignant tumor is a malignant tumor derived from the epiblast, for example a tumor selected from the group consisting of brain tumor, stomach cancer, lung cancer, pancreatic cancer, colorectal cancer, breast cancer, prostate cancer, endometrial cancer, ovarian cancer and leukemia.

The present invention also relates to the drug delivery system according to any one of items of the first aspect of the invention which is used in prophylaxis or treatment of a malignant tumor or in inhibition of growth, proliferation, migration or invasion of a tumor.

In one embodiment of the invention, the malignant tumor is a malignant tumor derived from the epiblast, for example a tumor selected from the group consisting of brain tumor, stomach cancer, lung cancer, pancreatic cancer, colorectal cancer, breast cancer, prostate cancer, endometrial cancer, ovarian cancer and leukemia.

The present invention also relates to a method of killing or damaging a malignant tumor stem cell or inhibiting growth, proliferation, migration or invasion of a tumor stem cell, comprising: administering to the stem cell an effective amount of the drug delivery system according to any one of items of the first aspect of the present invention, or the pharmaceutical composition according to any one of items of the third aspect of the present invention.

In one embodiment of the invention, the method is performed in vivo.

In one embodiment of the invention, the method is performed in vitro.

In one embodiment of the invention, the stem cell is selected from the group consisting of brain tumor stem cells, gastric cancer stem cells, lung cancer stem cells, pancreatic cancer stem cells, rectal cancer stem cells, breast cancer stem cells, prostate cancer stem cells, endometrial cancer stem cells, ovarian cancer stem cells and leukemia stem cells.

The present invention also relates to a use of the drug delivery system according to any one of items of the first aspect of the present invention or the pharmaceutical composition according to any one of items of the third aspect of the present invention in manufacture of a reagent, in which the reagent is used for killing or damaging a malignant tumor stem cell or inhibiting growth, proliferation, migration or invasion of a tumor stem cell.

In one embodiment of the invention, the reagent is used in an in vivo method.

In one embodiment of the invention, the reagent is used in an in vitro method.

In one embodiment of the invention, the stem cell is selected from the group consisting of brain tumor stem cells, gastric cancer stem cells, lung cancer stem cells, pancreatic cancer stem cells, rectal cancer stem cells, breast cancer stem cells, prostate cancer stem cells, endometrial cancer stem cells, ovarian cancer stem cells and leukemia stem cells.

The present invention also relates to the drug delivery system according to any one of items of the first aspect of the present invention, which is used in killing or damaging a malignant tumor stem cell or inhibiting growth, proliferation, migration or invasion of a tumor stem cell.

In one embodiment of the invention, it is used in an in vivo method.

In one embodiment of the invention, it is used in an in vitro method.

In one embodiment of the invention, the stem cell is selected from the group consisting of brain tumor stem cells, gastric cancer stem cells, lung cancer stem cells, pancreatic cancer stem cells, rectal cancer stem cells, breast cancer stem cells, prostate cancer stem cells, endometrial cancer stem cells, ovarian cancer stem cells and leukemia stem cells.

The present invention also relates to a kit for killing or damaging a tumor stem cell or inhibiting growth, proliferation, migration or invasion of a tumor stem cell, in which the kit comprises the drug delivery system according to any one of items of the first aspect of the present invention or the pharmaceutical composition according to any one of items of the third aspect of the present invention, and, optionally, further comprises an instruction for use thereof.

In the present invention, firstly, the drug molecule is loaded onto the surface of the carbon nanotube based on the non-covalent hydrophobic interaction between the hydrophobic carbon nanotube and the drug molecule, and then the modifying material is wrapped around and coated onto the surface of the drug-loaded carbon nanotube to improve their water solubility and biocompatibility, and finally the targeting molecule is bound to the modifying material at the outer layer to achieve active targeting to target cells.

In one embodiment of the present invention, the oxidized carbon nanotube is negatively charged by ionizing the functional groups such as carboxyl groups on the surface thereof, the drug molecule carrying negative charge is loaded by hydrophobic interaction, the electric potential of the oxidized carbon nanotube is further reduced, and the modifying material with positive charge and the targeting molecule with negative charge are respectively coated to the outer layer of the drug-loaded carbon nanotube via the layer-by-layer electrostatic self-assembly, thereby obtaining the carbon nanotube-drug delivery system.

In the present invention, a surface marker of cancer stem cell is used as a target, a carbon nanotube is selected and used as the basic carrier material, salinomycin and so on is used as anti-cancer stem cell drug, so as to construct a novel targeted drug delivery system, which can significantly inhibit proliferation of cancer stem cells, induce cancer stem cell apoptosis, penetrate into central necrotic zone of cancer stem cells. The present invention provides a novel strategy for the selective targeting and effective elimination of cancer stem cells, which is expected to fundamentally prevent the cancer recurrence and metastasis resulted from cancer stem cells.

The various aspects and features of the present invention are described in further details as below.

The various terms and phrases used herein have the same general meanings as known to those skilled in the art, and it is nevertheless desired that the present invention again specify and explain these terms and phrases in further details, and when the terms and phrases as mentioned have meanings different from those known in the art, the meanings expressed in the present invention shall prevail.

In the present invention, the term “carbon nanotube” has the meaning known in the art and is described, for example, in Iijima S., Nature, 1991, 354: 56.

In the present invention, the carbon nanotube is single-walled carbon nanotube or multi-walled carbon nanotube or a mixture of them in any ratio. In one embodiment of the present invention, the carbon nanotube is single-walled carbon nanotube.

For the carbon nanotube used in the present invention, its surface is bonded with a large number of functional groups such as carboxyl group, hydroxyl group or the like, or its surface chemical structure is modified by treatment of physical or chemical means. In one embodiment of the present invention, the carbon nanotube is treated with means such as smashing, sonication, ball milling, acidification, alkalisation or oxidation.

In one embodiment of the present invention, the carbon nanotube is an oxidized carbon nanotube. The method for preparation of an oxidized carbon nanotube is well known in the art. For example, carbon nanotubes can be treated with a mixture of concentrated acids. Through the treatment, active functional groups such as carboxyl groups and hydroxyl groups can be introduced at two ends and defects on side walls of the carbon nanotubes, and the length of carbon nanotubes can be shortened, which can be used for further functionalization of carbon nanotubes in the next step.

In the present invention, the term “length of carbon nanotubes” refers to a length generally expressed in statistical average. However, in some specific cases, for example, when observed under an electron microscope, the length of single carbon nanotube fiber is the length of single fiber. A typical example of measurement method for “the length of carbon nanotubes” is a microscope method, in particular, an electron microscope method.

In the present invention, unless otherwise indicated, the term “drug-loading capacity” as used herein refers to a percentage of the weight of drug molecule to the weight of carbon nanotube in the drug delivery system, that is, weight of drug molecule/weight of nanotube×100%.

In the present invention, the drug molecule can be adsorbed on the surface or cavity of the carbon nanotube.

In the present invention, the term “tumor stem cell (TSC)” is also referred to as “cancer stem cell (CSC)” and refers to a cell having self-renewal ability in a tumor and capable of producing heterogeneous tumor cells. The characteristics of cancer stem cells include self-renewal, high tumorigenicity, differentiation potential and drug resistance.

Cancer stem cells express a variety of cell surface markers, such as CD44, CD133, CD34, CD166, EpCAM. Among them, CD44 is a surface marker for gastric cancer stem cell, and is also highly expressed in other cancers such as breast cancer, brain tumors, pancreatic cancer. Usually, based on the nature of tumor markers, the tumor markers can be divided into seven categories: enzyme tumor markers, hormone tumor markers, embryonic antigen tumor markers, special protein tumor markers, glycoprotein antigen tumor markers, oncogene protein tumor markers and other tumor markers. In the present invention, the targeting molecule refers to a molecule capable of specifically binding to any of these cell surface markers.

In the present invention, the tumor and/or cancer includes, but is not limited to:

epithelial cell-derived tumors, including, but not being limited to, bladder cancer, breast cancer, colorectal cancer, renal cancer, liver cancer, lung cancer (including small cell lung cancer, non-small cell lung cancer), head and neck cancer, esophagus cancer, gallbladder cancer, gastric cancer, cervical cancer, ovarian cancer, thyroid cancer, prostate cancer and skin cancer (including squamous cell cancer);

hematopoietic tumors of lymphatic system, including but not being limited to leukemia, acute lymphocytic leukemia, acute lymphoblastic leukemia, B-cell lymphoma, T-cell lymphoma, Hodgkin lymphoma, non-Hodgkin lymphoma, hair cell lymphoma, mantle cell lymphoma, myeloma, and Burkitt's lymphoma;

hematopoietic tumors of bone marrow system, including but not being limited to acute and chronic myelogenous leukemia, myelodysplastic syndrome, and promyelocytic leukemia;

mesenchymal tumors, including but not being limited to fibrosarcoma and rhabdomyosarcoma;

tumors of central causes, including but not being limited to fibrosarcoma and rhabdomyosarcoma;

tumors of central and peripheral nervous system, including astrocytomas, fibroblastic neuromas, gliomas and schwannomas; and

other tumors, including but not being limited to melanoma, seminoma, teratocarcinoma, osteosarcoma, xenoderoma pigmentosum, thyroid cystocarcinoma, and Kaposi sarcoma.

In the present invention, the term “modifying material” refers to a material which has good biocompatibility and biodegradability and can be used for improving the water solubility of carbon nanotubes. Specific examples thereof include small molecular compounds with functional groups such as hydroxyl group, carboxyl group and amino group, macromolecular polymers such as polyethylene glycol, polyvinyl alcohol, sulfonated polyaniline and poly(propionylethyleneimine), as well as biological molecules such as amino acids and enzymes.

Salinomycin is a polyether antibiotic isolated from fermentation broth of Streptomyces albus. Its effect of killing breast cancer stem cells is more than 100 times that of paclitaxel which is a commonly used chemotherapy drug for breast cancer, and thus it can effectively inhibit growth and metastasis of breast cancer, and therefore is a selective inhibitor of breast cancer stem cells. Studies have shown that salinomycin is also very effective to gastric cancer stem cells, ovarian cancer stem cells, leukemia stem cells, endometrial cancer stem cells, lung cancer stein cells and colorectal cancer stem cells, indicating that salinomycin can be used as an anti-tumor stem cell drug. However, salinomycin is difficult to enter tumor tissues, does not have target ability, and has no selectivity between cancer stem cells and normal tissue stem cells, so that when killing cancer stem cells, it also causes inhibition and damage of function of normal stem cells and generates toxic and side effect. At the same time, salinomycin has poor water solubility, and its in vivo administration can only be carried out by intraperitoneal injection after being dissolved in ethanol, which greatly limits its application.

The drug delivery system of the present invention is particularly suitable for delivery of salinomycin.

The term “pharmaceutically acceptable salts” as used herein includes conventional salts formed from pharmaceutically acceptable inorganic or organic acids or inorganic or organic bases, and acid addition salts of quaternary ammoniums. More specific examples of suitable acid salts include salts of hydrochloric acid, hydrobromic acid, sulfuric acid, phosphoric acid, nitric acid, perchloric acid, fumaric acid, acetic acid, propionic acid, succinic acid, glycolic acid, formic acid, lactic acid, maleic acid, tartaric acid, citric acid, pamoic acid, malonic acid, hydroxyl maleic acid, phenylacetic acid, glutamic acid, benzoic acid, salicylic acid, fumaric acid, toluenesulfonic acid, methanesulfonic acid, naphthalene-2-sulfonic acid, benzenesulfonic acid, hydroxyl-2-naphthoic acid, hydroiodic acid, malic acid, steroic acid, tannic acid and the like. For other acids such as oxalic acid which per se are not pharmaceutically acceptable, they may be used to prepare salts useful as intermediates to obtain the compounds of the invention and pharmaceutically acceptable salts thereof. More specific examples of suitable base salts include salts of sodium, lithium, potassium, magnesium, aluminum, calcium, zinc, N,N′-dibenzylethylenediamine, chloroprocaine, choline, diethanolamine, ethanediamine, N-methylglucamine and procaine. When referring to pharmaceutically acceptable salts of the drug molecule of the invention, it generally refers to a salt of the drug molecule that is useful in the field of pharmaceutical production, is harmless to product or mammals, or has a reasonable or acceptable benefit/risk ratio.

As used herein, the term “derivative” refers to a compound in which an atom or radical of a drug molecule is substituted by other atom or radical, and which still has a comparable biological activity or an enhanced activity. Specifically, when salinomycin is taken as an example, its matrix can be substituted by alkyl such as methyl, ethyl and the like, and may also be substituted by a group such as halogen, hydroxyl, hydroxyalkyl, alkoxy, amino, alkylamino or the like. Thus, when a derivative is mentioned hereinafter, it generally refers to a drug molecule derivative that is useful in the pharmaceutical field, is harmless to the product or mammals, or has a reasonable or acceptable benefit/risk ratio.

The carbon nanotube-drug delivery system of the present invention can be administered in any manner known in the art, for example, in oral, intramuscular, subcutaneous administration and so on, and its dosage forms can be for example, tablets, capsules, buccal tablets, chewable tablets, elixirs, suspensions, transdermal agents, microencapsulated embedding agents, implants, syrups and the like, and can be common preparations, sustained-release preparations, controlled-release preparations and various microparticle drug delivery systems. In order to form tablets in unit dosage forms, various biodegradable or biocompatible carriers known in the art can be widely used. Examples of the carrier include, for example, saline aqueous solutions and buffered aqueous solutions, ethanol or other polyols, liposomes, polylactic acid, vinyl acetate, polyanhydrides, polyglycolic acid, collagen, polyorthoesters and the like.

The administration dosage of the carbon nanotube-drug delivery system of the present invention depends on many factors such as the nature and severity of disease to be prevented or treated, the gender, age, weight, sensitivity and individual response of patient or animal, the particular compound to be used, the route of administration, the number of doses administered, and the desired therapeutic effects. The above dosage may be administered in a single dosage form or divided into several dosage forms, for example, two, three or four dosage forms. The single maximum dose generally does not exceed 30 mg/Kg of body weight, for example 0.001-30 mg/Kg, preferably 0.01-5 mg/Kg, and preferably ranges 0.5-2 mg/Kg of body weight. However, in some cases, it is also possible to use a single dose of more than 30 mg/Kg of body weight or less than 0.001 mg/Kg.

### BRIEF DESCRIPTION OF THE DRAWINGS

The words in the drawings of the present invention have general meanings well known to those skilled in the art and, if not consistent with the well-known meanings, the meaning of the present invention prevails:

SAL: salinomycin

CHI: chitosan

HA: hyaluronic acid

SAL-SWNTs: salinomycin-loaded single-walled carbon nanotubes

Pristine-SWNT: pristine single-walled carbon nanotube

Ox/Oxidized-SWNTs: oxidized single-walled carbon nanotubes

SAL-SWNTs-CHI: chitosan-modified salinomycin-loaded single-walled carbon nanotubes

SAL-SWNTs-CHI-HA: hyaluronic acid/chitosan-modified salinomycin-loaded single-walled carbon nanotubes

Release rate

PE: percentage of expression

FITC: fluorescein isothiocyanate

Counts

FL2-Height: height of fluorescence pulse

Survival: survival rate

Isotype control

Free Mitomycin C: free mitomycin C

Free SAL: free salinomycin

Blank SWNTs-CHI-HA: blank hyaluronic acid/chitosan-modified single-walled carbon nanotubes

Control

PBS: phosphate buffer solution

AGS cell: human gastric cancer stem cell

Normal

Early apoptosis

Late apoptosis

Dead cells

Tumor spheroid volume ratio

FIG. 1 shows a process for preparation of SAL-SWNTs-CHI-HA in a specific embodiment of the present invention;

FIG. 2 shows the solubility and stability of functionalized carbon nanotubes in a PBS solution in a specific embodiment of the invention;

FIG. 3 shows a photo of transmission electron microscopy of functionalized carbon nanotubes in a specific embodiment of the present invention;

FIG. 4 shows the in vitro release behaviors of different salinomycin-loaded carbon nanotubes in a PBS solution at pH 7.4 in a specific embodiment of the present invention;

FIG. 5 shows the in vitro release behaviors of different salinomycin-loaded carbon nanotubes in a PBS solution at pH 5.5 in a specific embodiment of the present invention.

FIG. 6 shows the sorting, culturing and identification of gastric cancer stem cells in a specific embodiment of the present invention;

FIG. 6A shows the expression rate of CD44 in AGS gastric cancer cell lines as determined by flow cytometric analysis in a specific embodiment of the present invention: a1 is isotype control; a2 is gastric cancer stem cells stained with anti-CD44-FITC antibody;

FIG. 6B shows photographs of CD44+ cells (b1) and CD44− cells (b2) which are sorted from AGS cells and serum-free suspension cultured for 7 days in a specific embodiment of the present invention;

FIG. 6C shows phenotypic identification of suspended cell spheres in a specific embodiment of the invention: c1 is isotype control; c2 is gastric cancer stem cells stained with anti-CD44-FITC antibody;

FIG. 7 shows uptakes of gastric cancer stem cells in a specific embodiment of the present invention; in which,

FIG. 7A shows results of flow cytometry analysis in a specific embodiment of the present invention in which: 1 is Free HA+FITC-SWNTs-CHI; 2 is FITC-SWNTs-CHI; 3 is Free HA+FITC-SWNTs-CHI-HA; 4 is FITC-SWNTs-CHI-HA;

FIG. 7B shows confocal microscopy analysis in a specific embodiment of the present invention in which, a1 to a3 are FITC-SWNTs-CHI; b1 to b3 are FITC-SWNTs-CHI-HA; c1 to c3 are Free HA+FITC-SWNTs-CHI-HA, wherein 1 is nuclear staining; 2 is FITC staining; 3 is the result of superposition of 1 and 2;

FIG. 8 shows the inhibitory effects of three different salinomycin preparations and blank vector on CD44+ cells (FIG. 8A) and CD44− cells (FIG. 8B) in a specific embodiment of the invention;

FIG. 9 shows the effect of SAL-SWNTs-CHI-HA on self-renewal capacity of CD44+ cells in a specific embodiment of the present invention; in which,

FIG. 9A shows analysis of the expression rate of CD44 after different treatments;

FIG. 9B shows analysis of suspended cell spheres-forming ability;

FIG. 9C shows analysis of soft agar clone forming ability;

FIG. 10 shows the effects of SAL-SWNTs-CHI-HA on the migration and invasion of CD44+ cells in a specific embodiment of the present invention;

FIG. 10A shows analysis of scratch-repair capability;

FIG. 10B shows analysis of migration capability;

FIG. 10C shows analysis of invasion capability;

FIG. 11 shows the ability of different salinomycin preparations to induce apoptosis of gastric cancer stem cells in a specific embodiment of the present invention;

FIG. 12 shows the ability of various salinomycin preparations on penetration and inhibitory of gastric cancer stem cell spheres in a specific embodiment of the present invention;

FIG. 12A shows the ability of salinomycin preparations to penetrate the stem cell spheres as determined by laser confocal;

FIG. 12B shows the inhibitory effects of three different preparations of salinomycin on gastric cancer stem cell spheres.

### SPECIFIC MODELS FOR CARRYING OUT THE INVENTION

Embodiments of the invention will now be described in details in conjugation with the following examples, but it will be understood by those skilled in the art that the following examples are only illustrative of the invention and should not be considered as limiting the scope of the invention. If no specific conditions were specified in the examples, it was carried out under normal conditions or conditions recommended by the manufacturer. When the manufacturers of reagents or apparatus used were not indicated, they were conventional products commercially available.

| Chinese Name | English Name/specification | Manufacturer | Art. No. |
|---|---|---|---|
| Salinomycin | Salinomycin monosodium salt | Sigma-Aldrich Company of | 46729 |
| | hydrate | USA | |
| Single-walled carbon | tube diameter 1-2 nm, length 5-20 μm, | Beijing Nachen Science & | — |
| nanotubes | purity >95% | Technology Co., Ltd. | |
| Chitosan | Molecular weight: about 50000 Da | Sigma-Aldrich Company of | 448869 |
| | | USA | |
| Hyaluronic acid | Hyaluronic acid sodium salt | Sigma-Aldrich Company of | 96144 |
| | from Streptococcus equi; molecular | USA | |
| | weight: 70000-120000 Da | | |
| Human gastric cancer | — | Cell Bank of Typical Culture | — |
| AGS cells | | Preservation Commission, | |
| | | Chinese Academy of Sciences | |

### Example 1: Preparation of SAL-SWNTs-CHI-HA

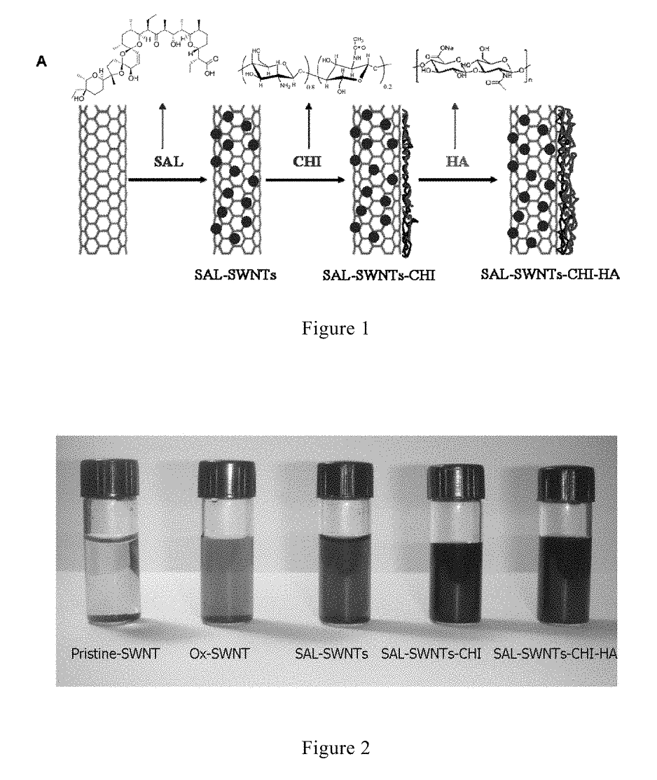

The preparation of SAL-SWNTs-CHI-HA was a relatively straight forward process, as shown in FIG. 1.

Commercial SWNTs could be purified and oxidized prior to modification of carbon nanotubes. Oxidation on the one hand was capable of removing impurities such as metal catalysts and amorphous carbon particles which had cell and tissue toxicity from carbon nanotubes, on the other hand could introduce active functional groups such as carboxyl groups, hydroxyl groups at both ends and side-wall defects of the carbon nanotubes, and could shorten the length of carbon nanotubes, thereby laying a foundation for the functionalization of carbon nanotubes in the next step.

For the preparation of SAL-SWNTs-CHI-HA, salinomycin was firstly loaded onto the surface of carbon nanotubes through the non-covalent hydrophobic interaction between hydrophobic SWNTs and salinomycin; then chitosan was wrapped around and coated onto surface of SAL-SWNTs to improve their water solubility and biocompatibility, and finally HA was bound to the external CHI layer to achieve the active targeting to CD44-expressing gastric cancer stem cells.

1. Preparation of SAL-SWNTs

Single-walled carbon nanotubes (SWNTs) were purified and oxidized by concentrated acid oxidation. Commercially available SWNTs (50 mg) were dispersed in 50 mL of concentrated H₂SO₄/HNO₃ (3:1, v/v) mixed acid and ultrasonicated at 40° C. for 12 h. After completion of the reaction, the reaction mixture was added to 1 L of deionized water, cooled, vacuum filtered through a ø0.10 μm nylon microporous filter with a Buchner filter apparatus, washed with deionized water until neutral, and then washed with 10 mM NaOH to remove oxidation fragments, and finally washed with deionized water to neutral, and lyophilized to obtain oxidized SWNTs.

3.0 mL of salinomycin methanol solution (concentration: 50 mg/mL) and 50 mg of oxidized SWNTs were mixed, ultrasonicated for 6 h, blow-dried with nitrogen, then, 5 mL of 0.01 M phosphate buffer solution (mixing 137 mmol NaCl, 2.7 mmol KCl, 8 mmol Na₂HPO₄, 2 mmol KH₂PO₄ and water, adjusting the pH to 7.4, replenishing with water to volume of 1 L) was added, ultrasonicated continuously for 6 h. The free salinomycin was removed by Φ 5.0 μm microfiltration membrane, and the filtrate was collected and washed with Φ 0.10 μm microfiltration membrane to obtain salinomycin-loaded carbon nanotubes (SAL-SWNTs).

2. Preparation of SAL-SWNTs-CHI

Chitosan was easy to combine with carbon nanotubes to improve the water solubility of carbon nanotubes, prolong the blood circulation time of carbon nanotubes and avoid the phagocytosis of reticuloendothelial system, so that the drug delivery system had more chance to reach the tumor tissues.

To 20 mL of 5 mg/mL chitosan aqueous solution (comprising 1% acetic acid), 20 mg SAL-SWCNTs were added, ultrasonicated at room temperature for 30 min, and then stirred overnight. The SAL-SWCNTs-CHI complex was obtained by washing for at least 5 times by centrifugation-ultrasonication-centrifugation method.

3. Preparation of SAL-SWNTs-CHI-HA

To 20 mL of 2 mg/mL hyaluronic acid aqueous solution, 20 mg of SAL-SWCNTs-CHI was added, subjected to ultrasonic treatment at room temperature for 30 minutes, and then stirred overnight. SAL-SWNTs-CHI-HA was obtained by washing for at least 5 times by centrifugation-ultrasonication-centrifugation method.

The results showed that the dispersity of SAL-SWNTs-CHI was still very good after standing at room temperature for 30 days due to surface coating with chitosan, while the oxidized SWNTs and SAL-SWNTs appeared obvious precipitates; in addition, the SAL-SWNTs-CHI-HA formed by modification of targeting molecule HA also had good water solubility and stability, as shown in FIG. 2.

4. Preparation of FITC-SWNTs-CHI-HA

SWNTs-CHI-HA labeled with fluorescein isothiocyanate (FITC, Sigma-Aldrich, Catalog No. F3651) was prepared as a fluorescent probe. FITC (dissolving 0.5 mg FITC in 1 mL acetone) was added to a solution of oxidized carbon nanotubes, and stirred overnight at 4° C. The reaction solution was subjected to collection by Φ 0.10 μm microfiltration membrane and washing to obtain FITC-SWNTs-CHI-HA complex.

5. Characterization of SAL-SWNTs-CHI-HA

The particle size and Zeta potential of SAL-SWNTs-CHI-HA were determined using a Nano Series Zen 4003 Zeta Sizer.

The drug-loading capacity of salinomycin in SAL-SWNTs was determined by spectrophotometry, in which methanol was used as desorbent, 4% vanillin solution was used as color developing agent, the color developing temperature was 60° C., the color developing time was 30 min, and the detection wavelength was 518 nm. The drug-loading capacity was calculated by the following formula:

$$Drug\_loading \_capacity ( \% ) = \frac{Mass\_of \_SAL \_loaded \_on \_SWNTs}{Mass\_of \_SWNTs + Mass\_of \_SAL \_loaded \_on \_SWNTs}$$

The results of particle sizes, Zeta potentials and drug-loading capacities of SAL-SWNTs-CHI and SAL-SWNTs-CHI-HA were shown in Table 1.

TABLE 1
Physical and chemical characterization of different
salinomycin-loaded carbon nanotubes

| | | | | Drug-loading |
|---|---|---|---|---|
| | Particle size | Polydispersity | Zeta potential | capacity, % |
| Formulation | (nm) | index | (mV) | (DLC, %) |
| Ox-SWNTs | 147.09 ± 1.06 | 0.35 ± 0.02 | −22.03 ± 1.46 | |
| SAL-SWNTs | 154.55 ± 5.31 | 0.26 ± 0.02 | −28.77 ± 3.88 | 32.74 ± 3.89 |
| SAL-SWNTs-CHI | 200.13 ± 1.72 | 0.38 ± 0.04 | 2.56 ± 0.2 | 26.29 ± 2.86 |
| SAL-SWNTs-CHI-HA | 237.09 ± 3.46 | 0.34 ± 0.03 | −11.23 ± 1.15 | 20.96 ± 1.62 |

The drug-loading capacities of SAL-SWNTs-CHI and SAL-SWNTs-CHI-HA were 26.29±2.86% and 20.96±1.62%, respectively. The results of Zeta-potential as measured further confirmed the modification process of SWNTs. The oxidized SWNTs had a surface potential of −22.03±1.46 mV due to the ionization of surface carboxyl groups. Further, after SAL with negative charge was loaded to the oxidized SWNTs, the potential was reduced to −28.77±3.88 mV, indicating that the anionic SAL was adsorbed on the sidewalls of the oxidized SWNTs. After functionalization with positively charged CHI, the potential of SAL-SWNTs-CHI increased to 2.56±0.20 mV. The potential of SAL-SWCNTs-CHI-HA obviously decreased to −11.23±1.15 mV, and it was confirmed that the negatively charged HA was coated onto the surface of SAL-SWCNTs-CHI by layer-by-layer electrostatic interaction.

The morphologies and structures of the pristine single-walled carbon nanotubes (SWNTs), the oxidized SWNTs, SAL-SWNTs, SAL-SWCNTs-CHI and SAL-SWCNTs-CHI-HA were observed by transmission electron microscopy.

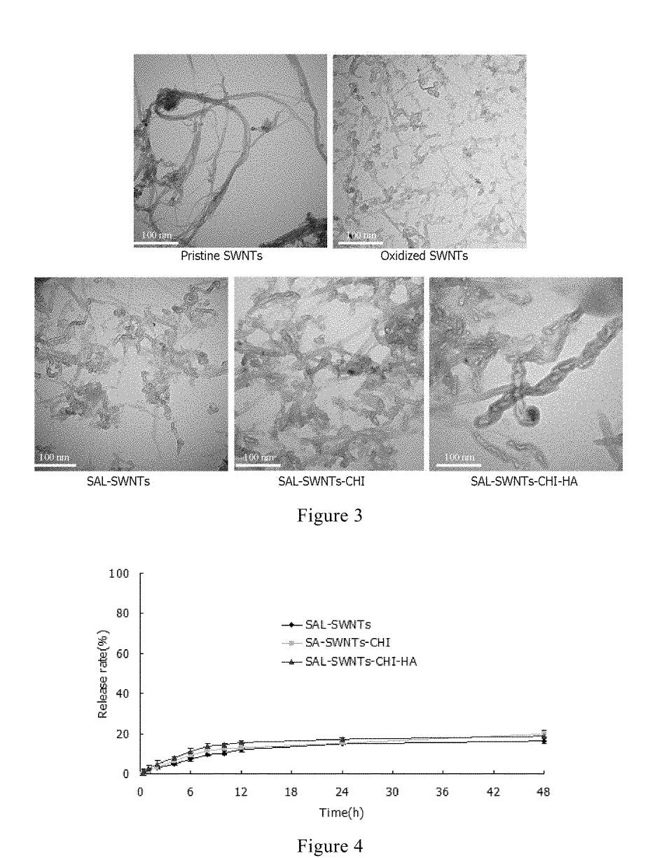

FIG. 3 shows the transmission electron microscopy results of functionalized SWNTs. It can be seen from the figure that pristine SWNTs were entwined with each other and aggregated because the pristine SWNTs were relatively long and had strong van der Waals interaction among the tubes. In comparison with the pristine SWNTs, the oxidized SWNTs were smooth and free of impurities, indicating that the oxidation treatment could remove metal particles and amorphous carbon. The oxidized SWNTs were significantly shortened, had better dispersability and had only aggregation of small bundles. Unlike the clean, smooth surface of the oxidized SWNTs, SAL-SWNTs had a rough SAL layer on the surface, confirming the presence of SAL on the surface of SWNTs. When chitosan was coated on the surface of SAL-SWNTs, the polysaccharide chains on the sidewalls of SWNTs were observed. In order to further introduce targeting molecules onto the surface of SWNTs, HA was coated on the CHI layer outside of the SAL-SWNTs-CHI by electrostatic self-assembly. As expected, the diameter of SAL-SWNTs-CHI-HA with bilayer of polysaccharide on the surface was significantly larger than that of SAL-SWNTs-CHI.

The in vitro release behaviors of the targeting salinomycin-loaded carbon nanotubes in phosphate buffered solution at pH 7.4 and pH 5.5 were determined by dialysis method.

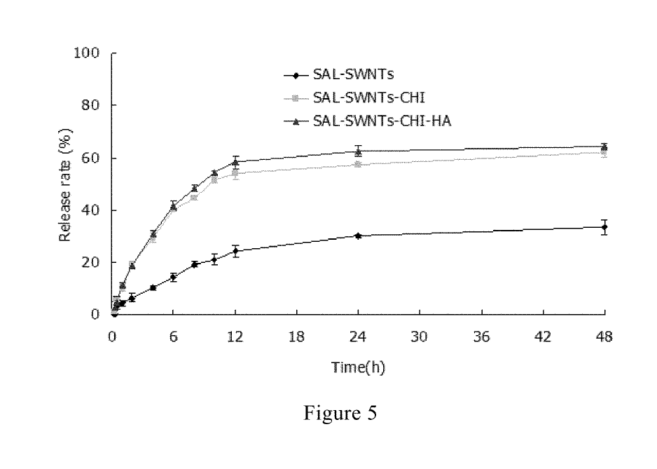

FIGS. 4 and 5 show the in vitro release behaviors of different salinomycin dosage forms at pH 7.4 (pH value of blood and normal tissues) and pH 5.5 (pH value of cell lysosomes and tumor tissues), respectively. The results showed that SAL-SWNTs-CHI and SAL-SWNTs-CHI-HA had similar cumulative release profiles. Three salinomycin-loaded carbon nanotubes released very slowly in PBS at pH 7.4, releasing only less than 20% of their own SAL after 48 hours; however, in environment of pH 5.5, the release rates of SAL increased significantly, in which both of SAL-SWNTs-CHI and SAL-SWNTs-CHI-HA released almost 60% of SAL after 12 h. This indicated that both drug delivery systems had pH-responsive properties for SAL release and provided the necessary conditions for intracellular delivery.

### Example 2: Sorting, Culturing and Identification of Gastric Cancer Stein Cells

It had been reported that CD44+ gastric cancer cells had characteristics of gastric cancer stem cells. In this study, gastric cancer stem cells were sorted from AGS gastric cancer cell lines by using cell surface marker CD44.

Cell culture and passaging: human origin gastric cancer AGS cells were cultured in DMEM/F12 (1:1) medium containing 10% fetal bovine serum and antibiotics (penicillin 100 U/ml and streptomycin 100 μg/ml) at 37° C. in a 5% CO₂ incubator. 0.25% trypsin was used as digestive solution to perform digestion and passage.

Sorting and culturing of gastric cancer stem cells: 0.25% trypsin was used to digest the AGS cells in logarithmic growth phase, individual cells were collected after digestion, washed with PBS for 2 times, adjusted to have a cell concentration of 1×10⁶/ml, added with antibody, anti-CD44-FITC, and incubated for 30 min at 4° C. Finally, the cells were washed twice with PBS and resuspended, passed through 40 μm cell sieves to ensure it was a single cell suspension. Before sorting, the cells were stored at 4° C. in dark. An isotype control antibody cell group was labeled under the same conditions. Before sorting by machine, propidium iodide (PI, with a final concentration of 1 μg/ml) was added to the cells of the experimental group and the control group respectively to exclude dead cells. The stained cells were sorted using a FACSDiva flow cytometer.

Culture and identification of gastric cancer stem cells: After sorting the AGS cells, the CD44+ cells were resuspended in serum-free DMEM/F12 medium (1% N2 (N2 additive, Gibco Company of USA, Catalog No. 17502-048), 2% B27 (B27 additive, Gibco Company of USA, Catalog No. 17504-044), 10 ng/mL bFGF (recombinant human basic fibroblast growth factor, Sigma-Aldrich Company of USA, Catalog No. F0291), 20 ng/mL EGF (epidermal growth factor, Sigma-Aldrich Company of USA, Catalog No. E9644)), and placed in a sterile low-adsorption 24-well culture plate at a density of 500/well and incubated in a 5% CO₂, 37° C. incubator. Medium change was performed every two days. When a large amount of suspended cell spheres appeared in the 24-well plate, the cells were collected, digested by adding trypsin, and single cells were obtained by gentle pipetting, and cultured in serum-free medium, so as to perform subculture of the suspended spheres. The percentage of CD44 expression in stem cells was detected by flow cytometry.

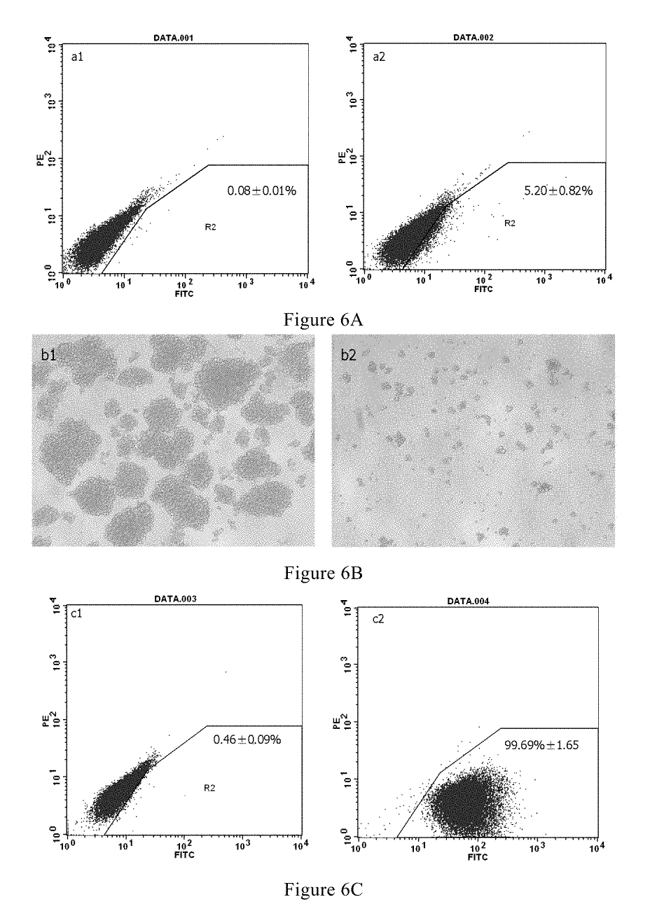

The results of immunofluorescence flow cytometry analysis showed that about 5.2±0.8% of the cells in human gastric cancer AGS cell line were gastric cancer stem cells (CD44+ cells) (see FIG. 6A).

The CD44+ cells and CD4− cells were suspension cultured in serum-free DMEM/F12 medium (1% N2, 2% B27, 10 ng/mL bFGF, 20 ng/mL EGF) to observe the formation of cell spheres, as shown in FIG. 6B. After one week of culture, the CD44+ cell group showed the formation of a large amount of cell spheres, while the CD44− cell group did not show significant formation of cell spheres, indicating that the CD44+ cells could be used as a model of gastric cancer stem cells.

FIG. 6C shows that the percentage of CD44+ cell subgroups could still reach 99.69% after flow sorting and suspension culture.

### Example 3: Targeting Ability of FITC-SWNTs-CHI-HA to Gastric Cancer Stein Cells

Flow cytometric analysis: CD44+ cells in an amount of 4×10⁵/well were seeded in a 6-well plate, cultured for 24 h, then incubated with FITC-SWNTs-CHI or FITC-SWNTs-CHI-HA (FITC final concentration of 5.0 μM) for 3 h at 37° C., respectively. After the completion of incubation, the cells were washed three times with cold PBS, after being digested with 0.25% trypsin, the cells were pipetted with PBS to form cell suspensions, and the cell-bound FITC fluorescence intensity was measured by flow cytometry (emission wavelength was 488 nm, detection wavelength was 520 nm). The number of cells used for each analysis was not less than 10⁵ and the number of cells collected was 10,000. The data were analyzed using FCS Express V3 software. In receptor competitive inhibition experiments, CD44+ cells were preincubated with excessive 5 mg/mL free HA for 30 min to saturate the CD44 receptors on the surface of CD44+ cells, and then incubated with FITC-SWNTs-CHI or FITC-SWNTs-CHI-HA (FITC final concentration was 5.0 μM) at 37° C. for 3 h and operated by the same method.

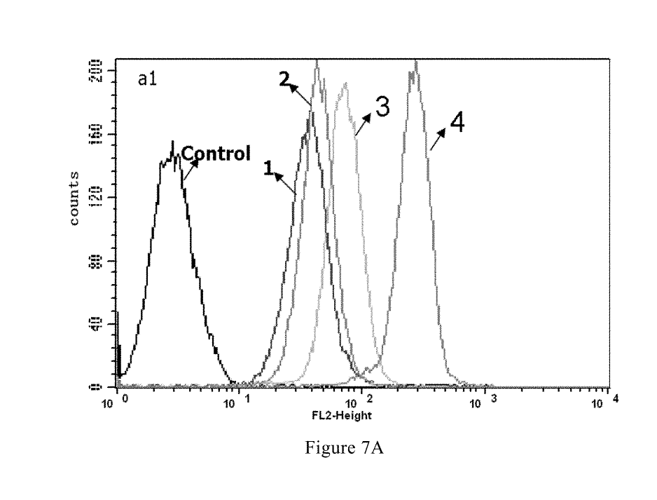

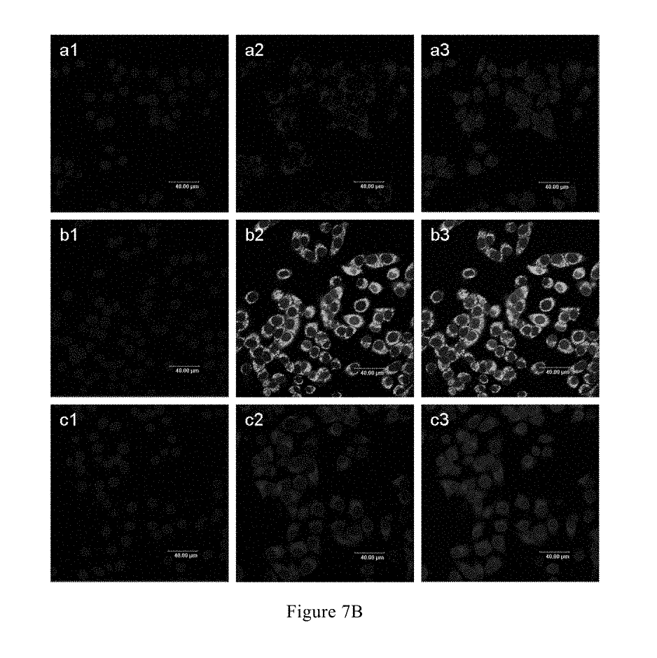

The flow cytometry results of gastric stem cell uptake indicated that the intracellular uptake to FITC-SWNTs-CHI-HA was significantly higher than that to FITC-SWNTs-CHI. In the competitive assay, free HA was used for pre-incubation with CD44+ cells for 30 min to saturate the surface CD44 receptors of CD44+ cells, the results showed that the CD44+ cells gave a significantly reduced uptake to FITC-SWNTs-CHI-HA, while the uptake to FITC-SWNTs-CHI did not significant change, as shown in FIG. 7A. This is due to the competitive binding of free HA to the CD44 receptors on the surface of CD44+ cells, which thereby reduced the binding of the HA on the surface of FITC-SWNTs-CHI-HA to the TF receptors on the surface of CD44+ cells. These results indicate that FITC-SWNTs-CHI-HA can specifically recognize CD44 receptors on the surface of CD44+ cells, thereby achieving active targeting to gastric cancer stem cells via receptor-mediated endocytosis.

Confocal Microscopy:

Laser confocal microscopy was used to determine the qualitative uptake to FITC-labeled carbon nanotubes by gastric cancer stein cells. The CD44+ cells were inoculated in a glass-bottom culture dish, and incubated in a 37° C., 5% CO₂ incubator for 24 h; added with FITC-SWNTs-CHI or FITC-SWNTs-CHI-HA (FITC final concentration was 5.0 μM), placed in carbon dioxide incubator, incubated at 37° C. for 3 h; rinsed three times with ice-cooled PBS, fixed with 4% paraformaldehyde for 10 min, then nuclear stained with 10 μM Hoechst 33258 (excitation wavelength was 352 nm, emission wavelength was 461 nm) for 30 min; rinsed with PBS three times. The images were analyzed by laser confocal microscopy. In receptor competitive inhibition experiments, CD44+ cells were preincubated with excessive 5 mg/mL free HA for 30 min to saturate the CD44 receptors on the surface of CD44+ cells, and then incubated with FITC-SWNTs-CHI or FITC-SWNTs-CHI-HA (FITC final concentration was 5.0 μM) at 37° C. for 3 h and operated by the same method.

FIG. 7B shows laser confocal analysis results of CD44+ cells with uptake of FITC-SWNTs-CHI, FITC-SWNTs-CHI-HA or free HA pre-saturated FITC-SWNTs-CHI-HA. The results showed that, as compared with FITC-SWNTs-CHI, the intracellular fluorescence of CD44+ cells administrated with FITC-SWNTs-CHI-HA was enhanced. The uptake of CD44+ cells to FITC-SWNTs-CHI-HA was significantly inhibited by pre-saturating CD44 receptors on the surface of CD44+ cells with free HA, leading to a decrease of intracellular fluorescence intensity (FIG. 7B, c1-c3), suggesting that FITC-SWNTs-CHI-HA was internalized into CD44+ cells via a CD44 receptor-mediated pathway. These results are consistent with the quantitative results of cellular uptake.

### Example 4: Inhibitory Effect of FITC-SWNTs-CHI-HA on Gastric Cancer Stein Cell Proliferation

CD44+ cells and CD44-cells sorted from human gastric cancer cell line AGS were seeded in an amount of 5000/well to 96-well plates, and incubated for 24 h at 37° C. in a 5% CO₂ incubator. Free salinomycin in a series of concentrations, SAL-SWNTs-CHI, SAL-SWNTs-CHI-HA or blank SWNTs-CHI-HA were added to, and same amount of drug-free culture medium was used as blank control. After the addition, the 96-well plates were incubated for 48 h at 37° C. in a 5% CO₂ incubator. After the completion of the cell culture, the plates were taken out and the culture media in the wells were removed. After washing with sterile PBS, 100 μL of PBS and 10 μL of WST-8 reagent were added to each well, and incubation was continued for 2 hours. Optical density (OD) was measured at the wavelength of 450 nm using a microplate reader. The toxicities of various salinomycin preparations on gastric cancer stem cells were evaluated by using the percentages of surviving cells (Survival rate, %) after the addition and culture. The percentages of surviving cells were calculated according to the following formula:

$$Cell\_survival \_rate , \% = \frac{OD\_value \_after \_drug \_treatment , {A}_{450 nm}}{OD\_value \_of \_blank \_control \_well , {A}_{450 nm}} \times 100 \%$$

Inhibition rate=1−Cell survival rate.

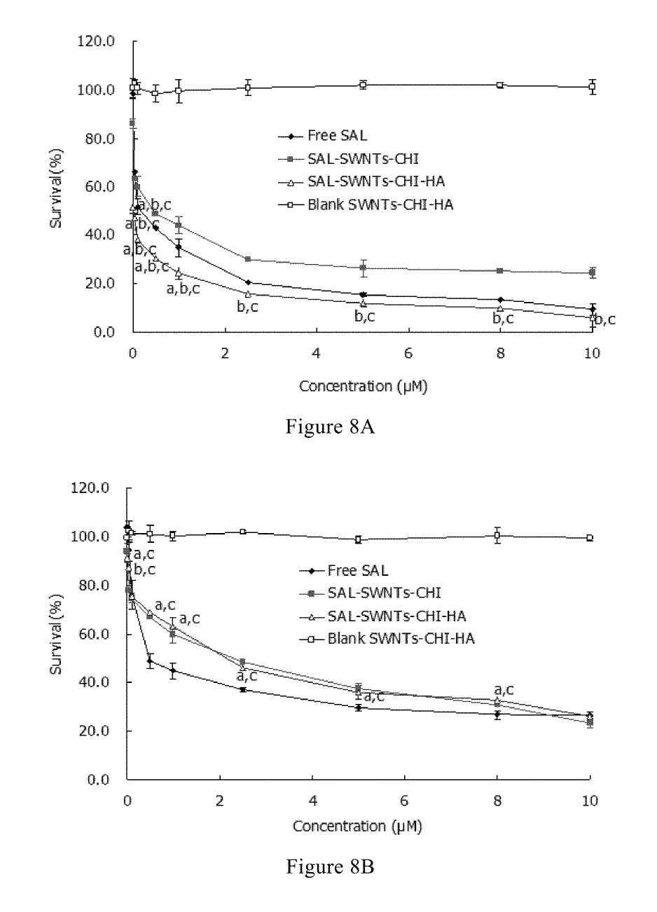

FIGS. 8A and 8B represent the inhibitory effects of different salinomycin preparations on CD44+ cells and CD44− cells, respectively. Compared to CD44− cells, all of free salinomycin and two salinomycin-loaded carbon nanotubes had strong inhibitory effects on the proliferation of CD44+ cells, indicating that gastric cancer stem cells were more sensitive to salinomycin than gastric cancer cells. Blank SWNTs-CHI-HA was non-toxic to CD44+ cells and CD44− cells even at high concentrations and could be used as drug delivery vehicles. Free salinomycin, SAL-SWNTs-CHI and SAL-SWNTs-CHI-HA had significant inhibitory effects on the proliferation of CD44+ cells, in which SAL-SWNTs-CHI-HA had the strongest inhibitory effect. As for CD44− cells, free salinomycin showed the strongest inhibitory effect, while SAL-SWNTs-CHI and SAL-SWNTs-CHI-HA had similar inhibitory effects due to the lack of receptor-mediated endocytosis.

### Example 5: Inhibitory Effects of FITC-SWNTs-CHI-HA on Self-Renewal Capacity of Gastric Cancer Stein Cells

The effects of SAL-SWNTs-CHI-HA on the self-renewal capacity of gastric cancer stem cells were studied by using CD44 expression rate, formation of suspended cell spheres, and formation of soft agar clones.

1. Effects on the Proportion of CD44+ Cells

In order to measure the effects of various salinomycin dosage forms on the expression of CD44 in AGS cells, AGS cells were seeded in 6-well plates at 3×10⁵ cells/well. After incubation for 24 h, AGS cells were incubated with free mitomycin, free salinomycin, SAL-SWNTs-CHI or SAL-SWNTs-CHI-HA (drug concentration was 1.0 μM) separately at 37° C. for 48 h. The blank medium was used as control. After incubation, the cells were washed three times with cold PBS, digested with 0.25% trypsin, and then pipetted with PBS to make cell suspensions. The expression rates of CD44 in AGS cells were detected by flow cytometry.

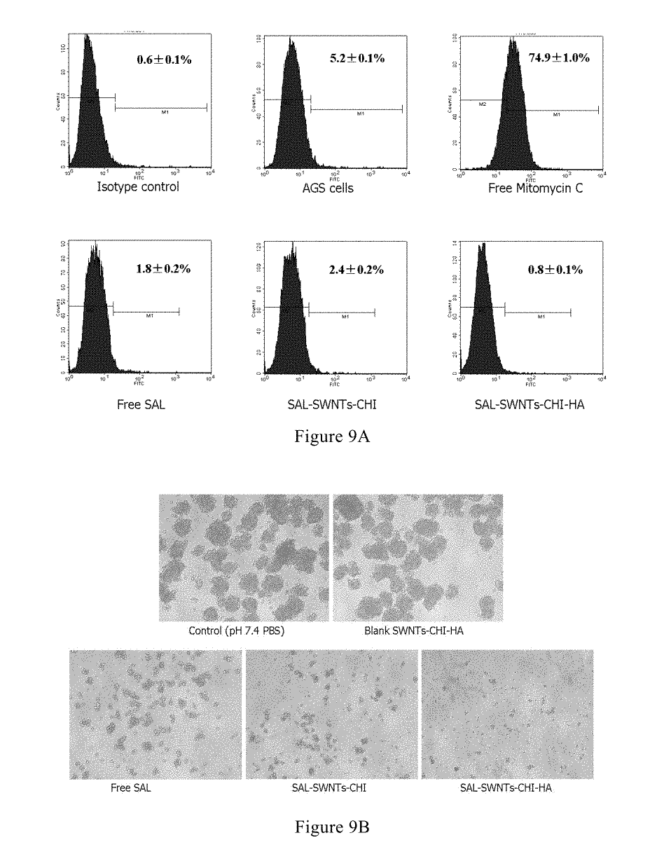

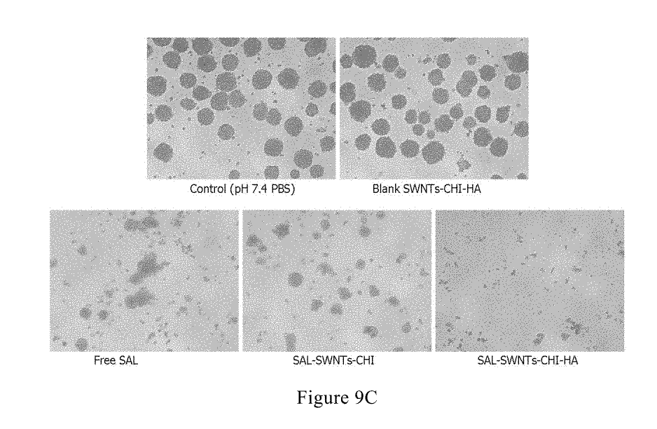

The effects of SAL-SWNTs-CHI-HA on the expression rates of CD44 in gastric cancer cells were shown in FIG. 9A. The proportion of CD44+ cells in the blank control group was 5.2±0.1%, and the proportion of CD44+ cells was significantly increased to 74.9±1.0% after treatment with mitomycin C, indicating that gastric cancer stem cells were highly tolerant to chemotherapeutic drugs. At the same time, the proportions of CD44+ cells decreased to 1.75±0.21%, 2.38±0.16% and 0.81±0.09%, respectively, after treatment with free SAL, SAL-SWNTs-CHI and SAL-SWNTs-CHI-HA, indicating that all SAL-containing dosage forms had selective toxicity to gastric cancer stem cells, in which SAL-SWNTs-CHI-HA had the strongest ability to eliminate gastric cancer stem cells.

2. Effects on Formation of Suspended Cell Spheres

Suspension cell culture technique was used to detect the effects of various salinomycin dosage forms on the ability of gastric cancer stem cells to form spheres. CD44+ cells were resuspended in serum-free DMEM/F12 medium (1% N2, 2% B27, 10 ng/mL bFGF, 20 ng/mL EGF) and placed in sterile low-adsorption 6-well plates with a density of 10000/well, separately added with PBS (pH 7.4, 0.1 M), blank SWNTs-CHI-HA, free salinomycin, SAL-SWNTs-CHI or SAL-SWNTs-CHI-HA (drug concentration was 0.5 μM), after incubated at 5% CO₂, 37° C. for 7 days, the formation of suspended cell spheres of each group was observed under an inverted microscope, and pictures were taken for recordation.

FIG. 9B represents the effects of SAL-SWNTs-CHI-HA on the ability of CD44+ cells to form suspended cell spheres. It was found that the blank SWNTs-CHI-HA vector had little effect on the ability of CD44+ cells to form suspended cell spheres as compared with the control, while all of salinomycin-containing dosage forms significantly reduced the number and size of the formed cell spheres, in which the CD44+ cells as treated with SAL-SWNTs-CHI-HA almost lost entire ability of forming cell spheres, indicating that SAL-SWNTs-CHI-HA could selectively inhibit the growth of gastric cancer stem cells.

3. Effects on Ability of Forming Soft Agar Clones

1.5 g of low-melting-point agar powder was placed in a conical flask, then added with 50 ml of deionized water, subjected to autoclaved sterilization, heated to melt agar before using, placed in a 50-55° C. water-bath for standby use; 3.0 ml of 3% agar maintained at 42° C. in molten state was taken, added to 12.0 ml of DMEM/F12 medium containing 10% FBS at 40° C., mixed and spread in 6-well plates at an amount of 1.5 ml per well, so as to form a bottom-layer gel with agar concentration of 0.6% at this time; 1 ml of 3% agar maintained at 42° C. in molten state was taken, added to 9 ml of DMEM/F12 culture medium containing 10% FBS at 39° C., and mixed to prepare an upper-layer culture medium having an agar concentration of 0.3%; CD44+ cells were digested with trypsin, then pipetted into single cell suspension and counted; the cell concentration was adjusted to 2×10⁵ cells/mL; 100 μl of the single cell suspension was taken and added to 2 ml of upper layer medium, mixed, gently spread on the fixed bottom-layer gel; PBS (PH 7.4, 0.1 M), blank SWNTs-CHI-HA, free salinomycin, SAL-SWNTs-CHI or SAL-SWNTs-CHI-HA (drug concentration was 0.5 μM) was added to each of the wells, respectively. After incubation in a 5% CO₂ incubator at 37° C. for 2 weeks, the formation of clones was observed under an inverted microscope and photos were taken for recordation. The above operations were repeated three times.

FIG. 9C represents the effect of SAL-SWNTs-CHI-HA on the ability of CD44+ cells to form soft agar clones. Similar to the results for the ability of forming suspended cell spheres, all of salinomycin-containing dosage forms significantly inhibited the ability of CD44+ cells to form soft agar clones, in which the SAL-SWNTs-CHI-HA had the strongest inhibitory effect, and the CD44+ cells as treated with SAL-SWNTs-CHI-HA almost completely lost the ability to form soft agar clones.

### Example 6: Inhibitory Effects of SAL-SWNTs-CHI-HA on Migration and Invasion of Gastric Cancer Stein Cells

The effects of SAL-SWNTs-CHI-HA on migration and invasion of gastric cancer stem cells were evaluated by scratch repair, Transwell migration and invasion assay.

1. Effects on Scratch Repair Capability

Scratch repair experiment was used to study the effects of various salinomycin dosage forms on the horizontal migration ability of gastric cancer stem cells. CD44+ cells were inoculated into 6-well plates in an amount of 1×10⁵ cells/well, and routinely cultured to reach 90% confluency. A 10 μl Tip head was used to scratch a straight line at the center of cells of each well. The cells were washed three times with PBS and added with fresh medium. Then, each of the wells was added with PBS (pH 7.4, 0.1 M), blank SWNTs-CHI-HA, free salinomycin, SAL-SWNTs-CHI or SAL-SWNTs-CHI-HA (drug concentration: 1.0 μM), photographed with a microscope in a state of 10× zoom. The cells were placed in a 37° C., 5% CO₂ incubator, and photographed again 24 hours after scratching. The differences of scratches healing between the various groups were observed.

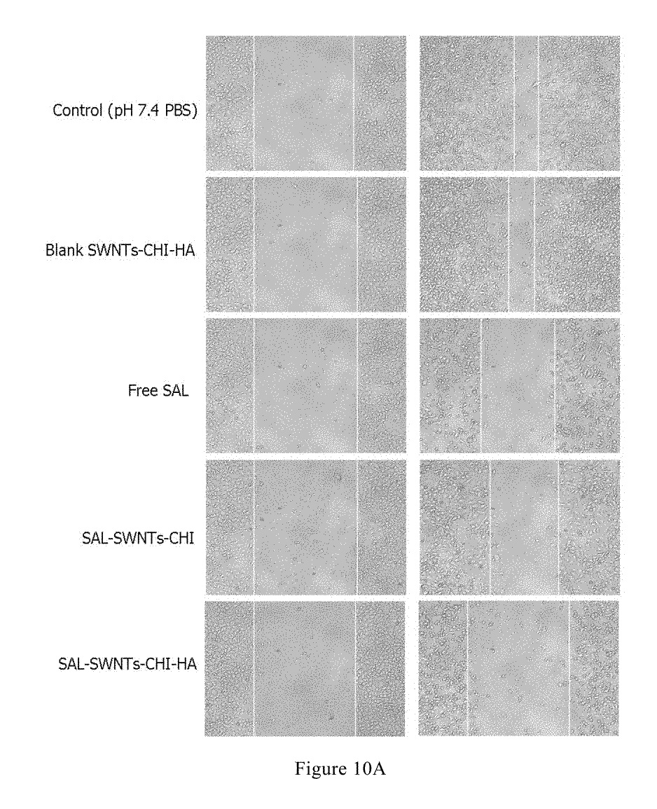

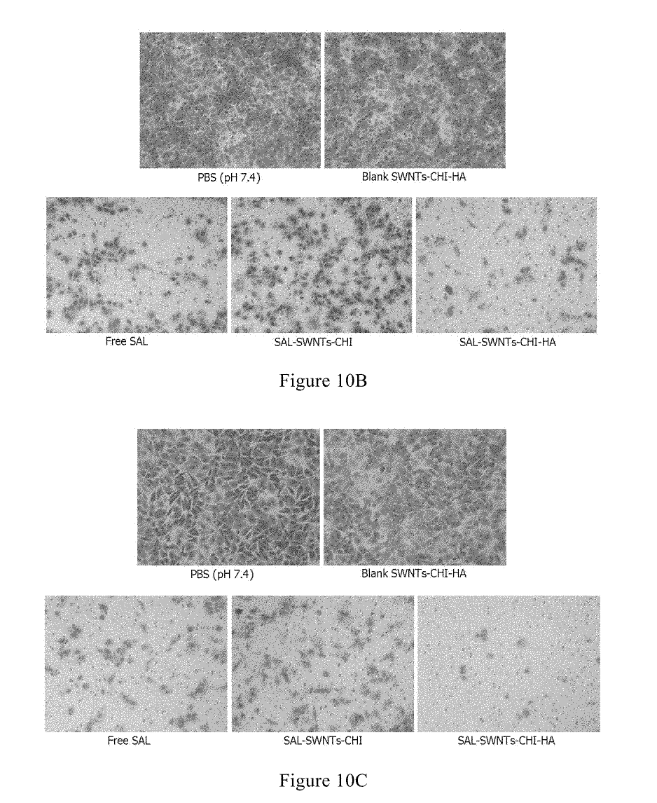

FIG. 10A shows the effects of different salinomycin dosage forms on the ability of gastric cancer stem cells to repair scratches. The results showed that the width of scratch at 24 h in the control group was only 22.5% of the original width at 0 h, and the scratch repair rate thereof was 77.5%. The blank SWNTs-CHI-HA vector had little effect on scratch repair rate. The SAL-SWNTs-CHI-HA almost completely inhibited the scratch repair ability of gastric cancer stem cells.

2. Effects on Migration Ability

Transwell migration assay was used to study the effects of various salinomycin dosage forms on the vertical migration ability of gastric cancer stem cells. Transwell cell compartments with pore diameter of 8 μm were placed in a 24-well plate. CD44+ stem cell spheres induced by serum-free culture at logarithmic growth phase were centrifuged at 1000 rpm for 3 min, and the cells were collected. The cells were then digested with 0.25% trypsin, pipetted to make single-cell suspension, and counted. The cells were inoculated into Transwell upper compartments in an amount of 100 μL, 5×10⁴ cells/well, and added with 100 μL of PBS (pH 7.4, 0.1 M), blank SWNTs-CHI-HA, free salinomycin, SAL-SWNTs-CHI or SAL-SWNTs-CHI-HA (drug concentration: 1.0 μM), respectively, the lower compartments were added with 800 μl of culture medium, incubated at 37° C. in a 5% CO₂ incubator. After 24 hours, the compartments were taken out, the uninvaded cells on bottom-gel and in the upper compartments were gently wiped with cotton swabs. The cells were then fixed with 4% paraformaldehyde for 20 minutes; washed with PBS three times, five minutes for each time; stained with Giemsa for 3 minutes; washed with distilled water three times; observed and photographed under microscope.

FIG. 10B shows the effects of SAL-SWNTs-CHI-HA on the migration ability of CD44+ cells. The results showed that SWNTs-CHI-HA had little effect on the migration ability of CD44+ cells as compared with the control group. The migration of CD44+ cells was significantly inhibited by three salinomycin dosage forms, and SAL-SWNTs-CHI-HA had the strongest inhibitory effect.

3. Effects on Invasive Ability

Transwell invasion assay was used to study the effects of various salinomycin dosage forms on the invasive ability of gastric cancer stem cells. Transwell cell compartments with pore diameter of 8 μm were placed in a 24-well plate. Matrigel gel, which had been previously dissolved and stored at 4° C. overnight, was taken, diluted with culture medium at a ratio of 1:2, gently placed in small chambers of 24-well plate, 30 μl/well, placed in a 37° C. incubator, and solidified after 2 hours. The subsequent invasion assay was performed in the same manner as the migration assay.

FIG. 10C shows the effects of SAL-SWNTs-CHI-HA on the invasion ability of CD44+ cells. The results showed that SWNTs-CHI-HA had almost no effect on the invasion ability of CD44+ cells as compared with the control group. All of the three salinomycin dosage forms significantly reduced the number of the invaded cells, in which SAL-SWNTs-CHI-HA had the strongest inhibitory effect.

### Example 7: In Vitro Induction of Apoptosis of Gastric Cancer Stem Cells

Flow cytometry was used to detect the apoptosis of gastric cancer stein cells via double-staining method with Annexin V-FITC and propidium iodide (PI), so as to observe the activities of various salinomycin dosage forms in induction of apoptosis of gastric cancer stem cells. CD44+ cells were seeded in a 6-well cell culture plate in an amount of 5×10⁵ cells/well (2 ml), and incubated at 37° C. in a 5% CO₂ cell incubator for 24 h; and separately added with PBS (pH 7.4, 0.1 M), blank SWNTs-CHI-HA, free salinomycin, SAL-SWNTs-CHI or SAL-SWNTs-CHI-HA (drug concentration: 5.0 μM), and continuously incubated at 37° C. for 12 h in a 5% CO₂ cell incubator. The 6-well cell culture plate was removed from the cell culture incubator, the supernatant was carefully sucked up; the cells were washed three times with cold pH7.4 PBS, collected and suspended in 200 μl of binding buffer. 5 μl of Annexin V-FITC and 5 μl of propidium iodide were added in the dark, placed in the dark at room temperature for 15 min, and the apoptotic rate of the cells was detected by flow cytometry.

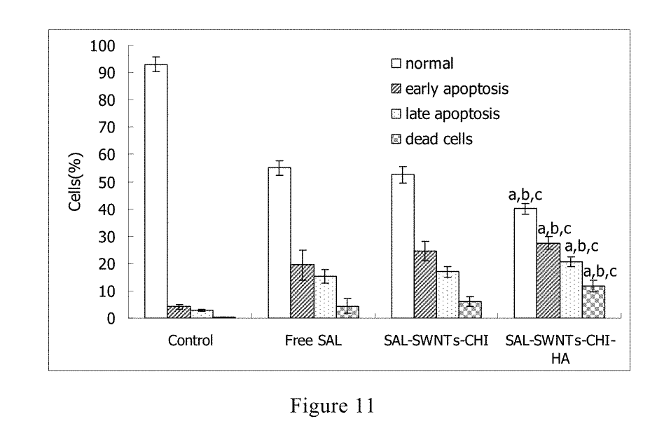

FIG. 11 shows the effects of different dosage forms of salinomycin on apoptosis of gastric cancer stem cells. The results showed that the apoptotic rates of gastric cancer stem cells were 34.8%, 39.4% and 47.8%, respectively, and the necrosis rates were 4.3%, 6.1% and 11.8%, respectively, after treatment with free SAL, SAL-SWNTs-CHI and SAL-SWNTs-CHI-HA, indicating that SAL-SWNTs-CHI-HA induced more apoptosis and necrosis of gastric cancer stem cells as compared to free SAL and SAL-SWNTs-CHI.

### Example 8: Inhibitory Effects of SAL-SWNTs-CHI-HA on Gastric Cancer Stein Cell Spheres

CD44+ cell suspension was inoculated to a low-absorption 24-well plate in an amount of 5×10⁴/well, and subjected to suspension culture with DMEM/F12 medium (1% N2, 2% B27, 10 ng/mL bFGF, 20 ng/mL EGF), incubated in a 5% CO₂ incubator at 37° C. for 6 days. Medium was replaced every three days. The stem cell spheres with diameter of more than 200 μm were transferred to a 96-well culture plate, one sphere per well.

1. Using Laser Confocal Microscopy to Observe the Ability of Salinomycin Preparations to Penetrate Stem Cell Spheres

Free FITC, FITC-SWNTs-CHI or FITC-SWNTs-CHI-HA were separately added to the wells of a 96-well plate containing stem cell spheres, and the concentration of FITC in the above preparations was 5 uM. After the addition, the 96-well plate was placed in a 37° C., 5% CO₂ incubator to continue the culture for 12 hours; then the stem cell spheres were transferred to a glass-bottom dish, 5 stem cell spheres in each group, washed three times with fresh culture medium, and then 100 μl of fresh culture medium was added in each dish; for each stem cell sphere, it was light-cut into layers from the top to the center of the sphere at 10 μm intervals, and FITC fluorescence intensities of different layers were studied.

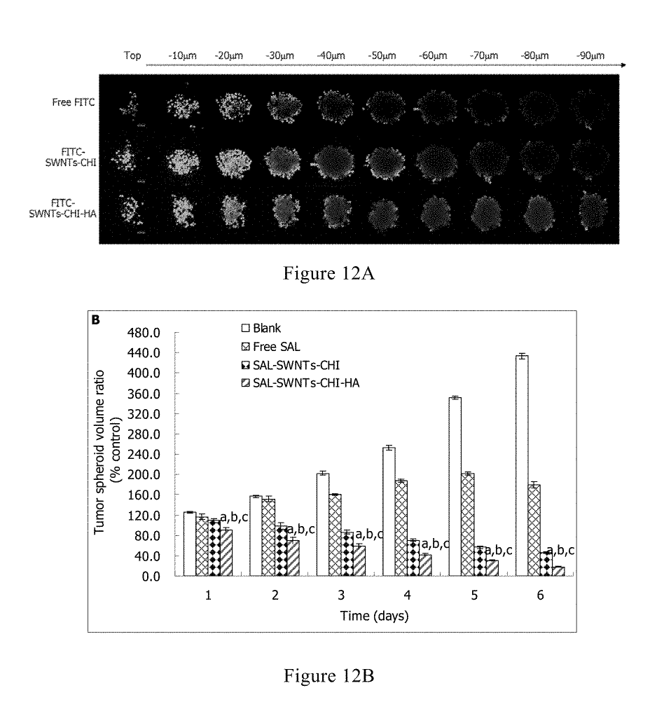

FIG. 12A shows laser confocal results of gastric cancer stem cell spheres hours after administration of various FITC dosage forms. After administration of free FITC, the gastric cancer stem cell spheres showed the weakest fluorescence intensity; after administration of FITC-SWNTs-CHI, FITC fluorescence was still not observed in the center of the cell spheres; while after administration of FITC-SWNTs-CHI-HA, strong fluorescence signals were observed in the whole cell spheres, indicating that it could penetrate into the center of the cell spheres.

2. Experiments of Inhibiting Stem Cell Growth

To the wells of 96-well culture plate, PBS, free salinomycin, SAL-SWNTs-CHI or SAL-SWNTs-CHI-HA were added, respectively, and the concentration of salinomycin in the above preparations was 5 μM. After addition, the 96-well plate was placed in a 37° C., 5% CO₂ incubator and incubated continuously. The growth of tumor spheres under these conditions was observed. The maximum and minimum diameters of the tumor spheres were recorded on the 1^{st}, 2^{nd}, 3^{rd}, 4^{th} and 5^{th} days after the administration. The formula for calculating the inhibition rate of tumor sphere growth was as follows: V=(π×dₘₐₓ×dₘᵢₙ)/6, in which dₘₐₓ was maximum diameter, dₘᵢₙ was minimum diameter; tumor sphere volume change rate %=(V_{dayi}/V_{day0}) 100%, in which V_{dayi} represents the volume of stem cell spheres on the i^{th} day after administration, and V_{day0} represents the volume of stem cell spheres before administration.

FIG. 12B shows the inhibitory effects of three different salinomycin dosage forms on gastric cancer stem cell spheres. On the 6^{th} day after administration of PBS, free SAL, SAL-SWNTs-CHI and SAL-SWNTs-CHI-HA, the volume change rates of cell spheres were 433.3±6.0%, 179.5±5.8%, 46.1±7.7% and 18.2±1.2%. Among all the preparations containing salinomycin, SAL-SWNTs-CHI-HA had the strongest inhibitory effect on the growth of gastric cancer stem cell spheres in vitro.

### CONCLUSION

In the present invention, aiming at anti-therapeutic mechanism of cancer stem cells, the nanometer material was used as a carrier to selectively deliver the anti-stem cell drug to the cancer tissue and penetrate into the cancer tissue; the targeting ligand molecule for the cancer cell-specific marker was linked to the nanometer carrier, so that the drug-delivery system that has entered into the cancer tissue could enter the cancer cell; the anti-stem cell drug was combined to the nanometer carrier, and thus it was effectively avoided that the drug was pumped out by transporter from the cancer cell; the sustained-release function of the nanometer carrier to anti-stem cell drug was utilized to maintain the drug in cancer cell at a high concentration level, so that the DNA repair capacity of the cancer stem cell was effectively impaired, and the apoptosis of the cancer stem cell was promoted.

The constructed SAL-SWNTs-CHI-HA, a cancer stem cell targeting drug delivery system, could significantly reduce the expression rate of CD44+ cells, the ability of forming suspending cell spheres and clones, the ability of migration and invasion and the growth of cancer stem cell spheres. These results suggest that SAL-SWNTs-CHI-HA can selectively remove cancer stein cells from cancer cell lines. The mechanism study showed that the receptor-mediated endocytosis of SAL-SWNTs-CHI-HA significantly enhanced the uptake of cancer stem cells to the drug as carried by SAL-SWNTs-CHI-HA, thereby inducing the apoptosis of cancer stem cells. This study will provide an effective strategy for the selective removal of cancer stem cell, thereby improving the treatment of cancer.

Although specific embodiments of the invention have been described in detail, those skilled in the art will understand that the technical solution of the present invention is not limited to the specific embodiments as described, but may include any combinations of the embodiments. Various modifications and substitutions may be made to those details in accordance with all teachings which have been disclosed and which are within the scope of the present invention. The full scope of the invention is given by the appended claims and any equivalents thereof.

What is claimed is:

1. A drug delivery system, comprising: a drug-loaded carbon nanotube formed by a carbon nanotube and a drug molecule adsorbed on the surface of the carbon nanotube by a non-covalent interaction, a modifying material capable of enhancing water solubility and biocompatibility of the drug delivery system, and a targeting molecule; wherein the modifying material is coated on the surface of the drug-loaded carbon nanotube by electrostatic self-assembly, so as to obtain a modified drug-loaded carbon nanotube, and the targeting molecule is coated on the surface of the modified drug-loaded carbon nanotube by electrostatic self-assembly.

2. The drug delivery system according to claim 1, wherein the drug molecule is loaded onto the surface of the carbon nanotube by hydrophobic interaction.

3.-8. (canceled)

9. The drug delivery system according to claim 1, wherein the drug-loaded carbon nanotube has a particle size of 130-200 nm, preferably 130-180 nm, and particularly preferably 130-160 nm.

10. The drug delivery system according to claim 1, wherein the drug-loaded carbon nanotube has a drug-loading capacity of 10 to 40% by weight, for example 15 to 30% by weight.

11. The drug delivery system according to claim 1, which has a particle size of 150-400 nm, for example 200-350 nm, for example 220-300 nm.

12.-16. (canceled)

17. The drug delivery system according to claim 1, wherein the drug molecule is a drug capable of specifically killing a tumor stem cell, such as salinomycin or a pharmaceutically acceptable salt or derivative thereof, parthenolide, sulforaphene, curcumin, resveratrol, metformin and so on.

18. The drug delivery system according to claim 1, wherein the modifying material is selected from the group consisting of a polymer macromolecule, a natural polysaccharide, a surfactant, an aromatic ring compound and a biological macromolecule; preferably, the modifying material is selected from the group consisting of chitosan, polyethylene glycol, a pluronic block polymer, celluloses, and preferably chitosan.

19. The drug delivery system according to claim 1, wherein the targeting molecule is a molecule capable of specifically targeting a cancer stem cell, for example selected from the group consisting of molecules capable of specifically targeting gastric cancer stem cells, breast cancer stem cells, endometrial cancer stem cells, lung cancer stem cells or colorectal cancer stem cells.

20. The drug delivery system according to claim 1, wherein the targeting molecule is selected from molecules that are capable of specifically binding to cellular markers on the surface of cancer stem cells, such as molecules capable of binding specifically to CD44, CD24, CD133, CD34, CD166 or EpCAM, for example is hyaluronic acid, P-selectin, or an antibody, for example a monoclonal antibody, capable of specifically binding to the cellular marker.

21. A method for preparing the drug delivery system according to claim 1, comprising the following steps:
(1) loading the drug molecule onto the surface of the carbon nanotube by a non-covalent interaction (e.g., π-π stacking interaction or hydrophobic interaction) to obtain the drug-loaded carbon nanotube;
(2) coating the modifying material onto the surface of the drug-loaded carbon nanotube by electrostatic self-assembly to obtain the modified drug-loaded carbon nanotube;
(3) adsorbing the targeting molecule to the surface of the modifying material by electrostatic self-assembly to obtain the drug delivery system;
preferably, the method further comprising: prior to step (1), a step of subjecting the carbon nanotube to oxidation treatment.

22. (canceled)

23. A pharmaceutical composition, comprising the drug delivery system according to claim 1, and a pharmaceutically acceptable carrier or excipient.

24.-27. (canceled)

28. A method of preventing or treating a malignant tumor or inhibiting growth, proliferation, migration or invasion of a tumor, the method comprising: a step of administering to a subject in need thereof the drug delivery system according to claim 1, or the pharmaceutical composition comprising the drug delivery system according to claim 1 and a pharmaceutically acceptable carrier or excipient.

29. The method according to claim 12, wherein the malignant tumor is a malignant tumor derived from epiblast, for example, a tumor selected from the group consisting of brain tumor, stomach cancer, lung cancer, pancreatic cancer, colorectal cancer, breast cancer, prostate cancer, endometrial cancer, ovarian cancer and leukemia.

30. The method according claim 12, wherein the subject is a mammal, such as a bovine, an equine, a goat, a porcine, a canine, a feline, a rodent, a primate animal; preferably, the subject is a human.

31. A method of killing or damaging a malignant tumor stem cell or inhibiting growth, proliferation, migration or invasion of a tumor stem cell, comprising: a step of administering to the stem cell an effective amount of the drug delivery system according to claim 1, or the pharmaceutical composition comprising the drug delivery system according to claim 1 and a pharmaceutically acceptable carrier or excipient.

32. (canceled)

33. The method according to claim 15, wherein the stem cell is selected from the group consisting of brain tumor stem cells, gastric cancer stem cells, lung cancer stem cells, pancreatic cancer stem cells, rectal cancer stem cells, breast cancer stem cells, prostate cancer stem cells, endometrial cancer stem cells, ovarian cancer stem cells and leukemia stem cells.

34.-39. (canceled)

40. A kit for killing or damaging a tumor stem cell or inhibiting growth, proliferation, migration or invasion of a tumor stem cell, wherein the kit comprises the drug delivery system according to claim 1 or the pharmaceutical composition comprising the drug delivery system according to claim 1 and a pharmaceutically acceptable carrier or excipient, and, optionally, further comprises an instruction for use.

41. The drug delivery system according to claim 1, wherein the non-covalent interaction is π-π stacking interaction or hydrophobic interaction.

42. The drug delivery system according to claim 1, having one or more of the following features:
(1) the carbon nanotube carries a negative charge;
(2) the drug molecule carries a negative charge;
(3) the modifying material carries a positive charge; and
(4) the targeting molecule carries a negative charge.

43. The drug delivery system according to claim 1, wherein the carbon nanotube has one or more of the following features:
(1) the carbon nanotube is single-walled carbon nanotube or multi-walled carbon nanotube;
(2) the carbon nanotube has a length of 100 to 1000 nm, preferably 150 to 400 nm;
(3) the carbon nanotube has an inner diameter of 1 to 3 nm, preferably 1 to 2 nm; and
(4) the carbon nanotube is an oxidized carbon nanotube;
optionally, the oxidized carbon nanotube has a particle size of 100 to 200 nm, preferably 100 to 150 nm, and particularly preferably 130 to 150 nm.
